(19) United States
(12) Patent Application Publication
(10) Pub. No.: US 2007/0265274 A1
(43) Pub. Date: Nov. 15, 2007

(54) 4-(4-METHYLPIPERAZIN-1-YLMETHYL)-N-[4-METHYL-3-(4-PYRIDIN-3-YL)PYRIMIDIN-2-YLAMINO)PHENYL]-BENZAMIDE FOR TREATING MUTATED-RET KINASE ASSOCIATED DISEASES

(76) Inventor: James Alexander Fagin, Cincinnati, OH (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/828,374

(22) Filed: Jul. 26, 2007

Related U.S. Application Data
(63) Continuation of application No. 10/521,927, filed on Feb. 1, 2006.
(60) Provisional application No. 60/398,409, filed on Jul. 24, 2002.

(51) Int. Cl. A61K 31/497; A61P 43/00

(52) U.S. Cl. 514/252.18

(57) ABSTRACT

The invention relates to the use of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof for the treatment of mutated-RET kinase associated disease, especially mutated-RET kinase associated thyroid cancer.

The invention relates to the use of 4-(4-methylpiperazin-1-ylmethyl)-N-[4-methyl-3-(4-pyridin-3-yl)pyrimidin-2-ylamino)phenyl]-benzamide (hereinafter: “COMPOUND I”) or a pharmaceutically acceptable salt thereof for the manufacture of a medicament for the treatment of a mutated-RET kinase associated disease, especially thyroid cancer harboring at least one mutation in the RET kinase, to the use of COMPOUND I or a pharmaceutically acceptable salt thereof in the treatment of a mutated-RET kinase associated disease, especially thyroid cancer harboring at least one mutation in the RET kinase, to a method of treating warm-blooded animals including mammals, especially humans suffering from a mutated-RET kinase associated disease, especially thyroid cancer harboring at least one mutation in the RET kinase by administering to a said animal in need of such treatment an effective dose of COMPOUND I or a pharmaceutically acceptable salt thereof.

The human RET gene, localized on chromosome 10q11.2 comprises 21 exons which encodes the protein RET kinase, a receptor tyrosine kinase (Takahashi M. and G. M. Cooper, 1987, Mol. Cell. Biol. 3:1378-1385). Receptor tyrosine kinases transduce the extracellular signal for processes as diverse as cell growth, survival and programmed cell death, differentiation and migration. The mature glycosylated protein is 170 kD in size, and contains three major domains: an extracellular domain involved in ligand binding that consists of cadherin-like and cysteine-rich regions; a transmembrane domain; and an intracellular portion containing the tyrosine kinase domain (TK) split by a 27 amino acid insertion.

The RET proto-oncogene is involved in the regulation of growth, survival, differentiation and migration of cells of neural crest origin. Four-ligands for the RET kinase have been identified: the glial cell line derived neurotrophic factor, neurturin, persephin, and artemin. After ligand binding, the RET kinase is induced to dimerize, resulting in activation of the kinase activity of the receptor, autophosphorylation at selected tyrosine residues, and initiation of intracellular signaling through interaction of effectors with specific tyrosine-phosphorylated domains of the receptor. The mutations in the RET gene involved in generation of either medullary thyroid cancer or papillary thyroid cancers code for constitutively active receptors in which one of the key regulatory functions that control its activation has been subverted. In sporadic papillary thyroid carcinomas rearrangements of RET resulting in constitutive activation of its tyrosine kinase function (RET/PTC) have been observed. This oncogenic hit is likely involved in disease causation, as demonstrated by the generation of papillary carcinomas in mice with targeted expression of RET/PTC in the thyroid by means of a thyroglobulin gene promoter.

Approximately 18,000 new cases of thyroid cancer are diagnosed each year in the USA. Of these, about 90% are papillary thyroid carcinomas (PTC) arising from thyroid follicular cells. Medullary thyroid carcinomas (MTC) originate from calcitonin-secreting parafollicular C cells, and represent 5 to 10% of all thyroid cancers.

A variable proportion of sporadic and radiation-induced papillary thyroid carcinomas (PTCs) were found to have somatic translocation involving the 3′ half of the RET kinase containing the tyrosine kinase (TK) and the 5′ end of other genes. The fusion proteins resulting from those rearrangements usually allow the constitutive activation of the RET tyrosine kinase leading to PTC formation.

As many as 75% of all medullary thyroid carcinomas are sporadic and about 25% of medullary thyroid carcinomas are hereditary, either as part of multiple endocrine neoplasia type 2 (MEN2), or of familial medullary thyroid carcinoma (FMTC). Germline mutations of the RET proto-oncogene confer predisposition to all hereditary forms of MTC, through an autosomal dominant mode of transmission.

The hereditary form of MTC, MEN 2, is divided into three subtypes depending on the organs involved. Multiple endocrine neoplasia comprises MTC, pheochromoctyoma (PC) in approximately 50% of the cases and hyperparathyroidism in 15 to 30% of the cases. MEN type 2A is the most common likely accounting for more than 90% of all MEN cases. Analysis of RET in MEN2A and FMTC families revealed germline mutations in affected individuals but not in unaffected individuals or normal controls. In each case, one of five particular cystein codons in exon 10 (C609, C611, C618, C620, C790) or V804 or exon 11 (C634) was found to be mutated. Mutations were detected in 98% of unrelated classic MEN 2A families and were found in 85% of FMTC families. In MEN 2B, single point mutations have been identified: amino acid 918 in exon 16 in 95% of the cases, amino acid 883 or amino acid 922.

4-(4-methylpiperazin-1-ylmethyl)-N-[4-methyl-3-(4-pyridin-3-yl)pyrimidin-2-ylamino)phenyl]-benzamide hereinafter referred as COMPOUND I, has the following formula 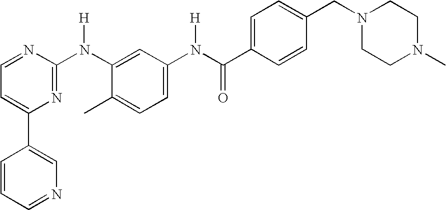
COMPOUND I free base and its acceptable salts thereof are disclosed in the European Patent application 0564409.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid.

COMPOUND I mesylate, herein after denominated “SALT I” and COMPOUND I mesylate alpha and beta crystal forms are disclosed in International Patent application WO 99/03854 published on January 1999.

Surprisingly, it has now been found that COMPOUND I, e.g. SALT I, can be used as a therapeutic agent for the treatment of a mutated-RET kinase associated disease, especially in thyroid cancer harboring at least one mutation in the RET kinase.

COMPOUND I or a pharmaceutically acceptable salt thereof, e.g. SALT I, inhibits in vitro the growth of mutated-RET kinase transformed fibroblasts. The autophosphorylation of the RET kinase-fusion protein (RET rearrangement such as RET/PTC1 and RET/PTC3) and the phosphorylation of phospholipase C gamma (PLCgamma downstream effector of the RET kinase) are inhibited by COMPOUND I or a pharmaceutically acceptable salt thereof, e.g. SALT I.

Hence, the invention relates to a method of treating a warm-blooded animal having a mutated-RET kinase associated disease, especially thyroid cancer harboring at least one mutation in the RET kinase, comprising administering to said animal in need of such a treatment COMPOUND I or a pharmaceutically acceptable salt thereof, e.g. SALT I, in a quantity which is therapeutically effective against said disease.

The invention relates to a method for administering to a human subject suffering from a mutated-RET kinase associated disease, especially in thyroid cancer harboring at least one mutation in the RET kinase, COMPOUND I or an acid addition salt thereof and preferably the monomethanesulfonate salt of 4-(4-methylpiperazin-1-ylmethyl)-N-[4-methyl-3-(4-pyridin-3-yl)pyrimidin-2-ylamino)phenyl]-benzamide.

RET rearrangements are particularly common in pediatric papillary thyroid carcinomas.

In one embodiment, the present invention provides in particular a method of treating pediatric thyroid carcinomas.

In another embodiment, the present invention provides a method of treating thyroid cancers caused by exposure to radiation.

The term “a mutated RET kinase-associated disease” as used herein includes but is not restricted to the following diseases:

thyroid cancers

breast cancers

other neoplasms associated with activation of the RET oncogene, e.g. tumors of the adrenal medulla (pheochromocytoma (PC)) and mucosal neuromas,

hyperparathyroidism (HPT) and parathyroid hyperplasia,

Hirschsprungs disease

Cutaneous Lichen amyloidosis

The term “thyroid cancer” as used herein comprises, but is not restricted to, e.g., multiple endocrine neoplasia of type 2 (MEN2), medullary thyroid carcinomas or papillary thyroid carcinomas and anaplastic thyroid cancer.

Preferably, COMPOUND I or a pharmaceutically acceptable salt thereof is used for the treatment of multiple endocrine neoplasia of type 2 (MEN2), medullary thyroid carcinomas or papillary thyroid carcinomas.

According to the invention, COMPOUND I or a pharmaceutically acceptable salt thereof is used for the treatment of thyroid cancer different from anaplastic thyroid cancer.

The term “treatment” comprises the administration of COMPOUND I or a pharmaceutically acceptable salt thereof, e.g. SALT I, to a warm-blooded animal in need of such treatment with the aim to cure the tumor or to have an effect on tumor regression or on the delay of progression of a disease.

The term “delay of progression” as used herein means that the tumor growth or generally, the disease progression is at least slowed down or hampered by the treatment and that patients exhibit higher survival rates than patients not being treated or being treated with placebo.

The term “a mutated-RET kinase” includes but is not restricted to RET kinase protein having at least a point mutation in a codon, a gene rearrangement leading to a fused protein or a disregulated expression.

The pharmaceutical compositions according to the present invention can be prepared in a manner known per se and are those suitable for enteral, such as oral or rectal, and parenteral administration to warm-blooded animals, including man, comprising a therapeutically effective amount of at least one pharmacologically active ingredient, alone or in combination with one or more pharmaceutically acceptable carries, especially suitable for enteral or parenteral application. The preferred route of administration of the dosage forms of the present invention is orally.

The person skilled in the pertinent art is fully enabled to select relevant test models to prove the beneficial effects mentioned herein on a mutated-RET kinase associated disease, especially in thyroid cancer harboring at least one mutation in the RET kinase. The pharmacological activity of such a compound may, for example, be demonstrated by means of the Examples described below, by in vitro tests and in vivo tests in nude or transgenic mice or in suitable clinical studies. Suitable clinical studies are, for example, open label non-randomized, dose escalation studies in patients with metastatic medullary thyroid carcinoma. The efficacy of the treatment is determined in these studies, e.g., by evaluation of the tumor sizes every 6 weeks by suitable serum tumor markers or by scintigraphy tumor detection with the control achieved on placebo matching with the active ingredient.

The effective dosage of COMPOUND I or a pharmaceutically acceptable salt thereof, e.g. SALT I, may vary depending on the particular compound or pharmaceutical composition employed, on the mode of administration, the type of the thyroid cancer being treated or the severity of the thyroid cancer being treated. The dosage regimen is selected in accordance with a variety of further factors including the renal and hepatic function of the patient. A physician, clinician or veterinarian of ordinary skill can readily determine and prescribe the effective amount of compounds required to prevent, counter or arrest the progress of the condition.

Depending on species, age, individual condition, mode of administration, and the clinical picture in question, effective doses of COMPOUND I or a pharmaceutically acceptable salt thereof, e.g. SALT I, for example daily doses corresponding to about 10-1000 mg of the active compound (free base), preferably 50-600 mg, especially 100 to 400 mg, are administered to warm-blooded animals of about 70 kg bodyweight. For adult patients with thyroid cancer or a related disease, a starting dose of 200 or 400 mg daily can be recommended. The daily doses for a juvenile are of 100-400 mg/m² of body surface, most preferably 340 mg/m². For patients with an inadequate response after an assessment of response to therapy, dose escalation can be safely considered and patients may be treated as long as they benefit from treatment and in the absence of limiting toxicities.

The present invention relates also to a method for administering to a human subject suffering from a mutated-RET kinase associated disease, especially in thyroid cancer harboring at least one mutation in the RET kinase, COMPOUND I or a pharmaceutically acceptable salt thereof, which comprises administering a pharmaceutically effective amount of COMPOUND I or a pharmaceutically acceptable salt thereof to the human subject, e.g., once daily, e.g. for a period exceeding 3 months. The invention relates especially to such method wherein a daily dose of 50 to 600 mg, preferably 100 to 400 mg is administered to an adult and 200 to 400 mg/m² of body surface to a juvenile, most preferably 340 mg/m² of body surface.

### EXAMPLES

Cell Line: PCCL3, a rat thyroid cell line, is maintained in H4 complete medium consisting of Coon's medium/F12 high zinc supplemented with 5% FBS, 0.3 mg/ml L-glutamine, 1 mU/ml TSH, 10 μg/ml insulin, 5 μg/ml apo-transferrin, 10 nM hydrocortisone, and penicillin/streptomycin. The expression system used was developed by Bujard and co-workers to deliver doxycyclin-inducible expression based on the high specificity of interactions of the E. coli tet repressor-operator with doxycyclin. Stable transfections are performed first to establish clonal lines constitutively expressing the transactivator rtTA (composed of a fusion of the rtetR DNA binding domain and the VP16 activation domain). Individual rtTA-expressing clones are then explored for doxycyclin-inducible expression by transient transfection with a luciferase reporter construct under control of a tet-operator. Clones of rtTA demonstrating very low or undetectable basal luciferase activity and marked induction (i.e. >100 fold) by doxycyclin are selected as hosts for secondary stable transfection with constructs consisting of a minimal CMV promoter containing tet-operator sequences cloned upstream of either RET/PTC1 or RET/PTC3 cDNAs.

RET/PTC1 and RET/PTC3 result from chromosomal rearrangements linking the promoters of unrelated genes to the C-terminal fragment of the RET kinase that is missing the extracellular and transmembrane domains. This rearrangement results in the production of a truncated form of the RET kinase being constitutively activated.

The most common germline mutation of the RET kinase is the amino acid 634-cysteine being mutated which leads to the constitutive dimerization and activation of the receptor.

### Example I

### In Vitro Kinase Reactions

Confluent T-75 flasks of RET/PTC3 (PCCL3) cells incubated with or without doxycycline for 24 hours are washed with ice cold PBS containing 0.2 mM sodium ortho-vanadate. Cells are lysed with ice-cold RIPA buffer (20 mM Tris pH 7.4, 150 mM NaCl, 1% Nonidet P-40, 1% Tween 20, 20 mM sodium fluoride, 1 mM sodium ortho-vanadate, 1 mM EGTA, 5 mM EGTA, 0.2 mM PMSF and Sigma Protease inhibitor mix) with constant agitation at 4° C. for 20 min. Cell lysates are passed through a 26-gauge needle to disperse large aggregates, and centrifuged for 20 min at 10,600 g at 4 C to yield the total cell lysate. The cleared supernatants are incubated with anti-RET kinase antibody (Santa Cruz goat polyclonal) for 2 hours at 4° C. and then incubated with Protein AG agarose (Santa Cruz) previously washed with RIPA buffer followed by an additional incubation at 4° C. for 90 min. The immune-complexes are spun with two washes in washing buffer (50 mM HEPES pH 7.2, 20 mM MnCl₂, 5 mM MgCl₂) and one final wash with kinase buffer (washing buffer plus 0.5 mM dithiotreitol). Kinase assays are performed in 20 μl of incubation buffer containing DMSO (0.5%), or inhibitor diluted in DMSO. The reactions in duplicate are performed by addition of ATP mix containing γP³²-ATP (Perkin-Elmer >6000 Ci/mmol) with specific activity of 140 nCi/pmol and incubated for 25 min at room temperature. Reactions are stopped by two washes with STOP Buffer (10 mM phosphate buffer, 1% TritonX-100, 0.1% sodium desoxycholate, 1 mM sodium ortho-vanadate, 1 mM ATP, 5 mM EDTA, 5 μg/ml aprotinin). After the second wash, the reactions are boiled in 35 μl Laemmli buffer for 10 min, the proteins are subjected to SDS-PAGE (7.5%) and their phosphorylation measured by PhosphoImager densitrometry (Molecular Dynamics, Sunnyvale, Calif.) after transfer to nitrocellulose membranes. Protein loading is then normalized to total RET kinase protein determined by Western blot analysis using either the goat polyclonal anti-RET kinase antibody (Santa Cruz) or a mouse monoclonal (University of Cincinnati).

100 nM of SALT I inhibits autophosphorylation of RET/PTC3 by 40%.

### Example 2

### Effect of SALT I on Growth of NIH3T3 Cells Expressing Constitutively Active RET MEN2A

The RETC634 mutation is the most common germline mutation of the RET kinase occurring in 85% of multiple endocrine neoplasias type 2A. The effect of SALT I on growth of NIH3T3 cells stably expressing a constitutively active form of the RET kinase (RETC634 mutation) is investigated.

NIH3T3 cells expressing constitutively active RET MEN2A are allowed to plate overnight in 6-well plates. They are then grown in the presence of 1 or 5% serum with SALT I 500 nM or with a vehicle solvent (control) for 9 days, with media changes every 3 days. Cells are counted after harvesting with EDTA/trypsin solution on day 9 after initiation of treatments.

| | | |
|---|---|---|
| serum | 1% | 5% |

| | | | | | |
|---|---|---|---|---|---|
| conditions | control vehicule | + | + | + | + |
| | SALT I 500 nM | − | + | − | + |

| | | | | |
|---|---|---|---|---|
| % of cell decrease | 0 | 42 | 0 | 15 |

SALT I inhibits the growth of RET kinase-transformed NIH3T3 fibroblasts.

### Example 3

### Effects of SALT I on Activation of (Phospholipase C) PLCγ by RET/PTC

The RET kinase associates with and phosphorylates PLCγ. To further explore the effect of SALT I on RET kinase activity, pretreatment with SALT I on PLCγ phosphorylation is examined.

Ret-PTC3 cells are seeded at 10⁵ cells/well in 6-well Corning plates. After 3 days, cells are treated with or without doxycycline in the presence of 250 nM of SALT I for 24 h. Cells are rinsed twice with cold (phosphate buffered saline) PBS containing 0.1 mM sodium orthovanadate, and left for 20 minutes in ice-cold RIPA buffer shaking gently at 4° C. Cell lysates are collected by centrifugation at 10,600 g for 20 min at 4° C. Protein assays are performed on aliquots of supernatants by the Coomassie Blue assay (Pierce, Rockford, Ill.). Western blot analysis are performed by running 100 μg of protein on SDS PAGE (5%), transfer to nitrocellulose membrane and probing initially with anti-phospho PLCγ antibody (Cell Signaling) and then with anti-PLCγ (Cell Signaling) for normalization.

In the presence of 250 nM of SALT I, the inhibition of phosphorylation of PLCγ is 28% in comparison with samples without SALT I.

### Example 4

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate β-crystal form

Capsules containing 119.5 mg of SALT I corresponding to 100 mg of COMPOUND I (free base) as active substance are prepared in the following composition:

| Composition | | |
|---|---|---|
| SALT I | 119.5 | mg |
| Cellulose MK GR | 92 | mg |
| Crospovidone XL | 15 | mg |
| Aerosil 200 | 2 | mg |
| Magnesium stearate | 1.5 | mg |
| | 230 | mg |

The capsules are prepared by mixing the components and filling the mixture into hard gelatin capsules, size 1.

### Example 5

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate, β-crystal form

Capsules containing 119.5 mg of SALT I corresponding to 100 mg of COMPOUND I (free base) as active substance are prepared in the following composition:

| Composition | | |
|---|---|---|
| Active substance | 119.5 | mg |
| Avicel | 200 | mg |
| PVPPXL | 15 | mg |
| Aerosil | 2 | mg |
| Magnesium stearate | 1.5 | mg |
| | 338.0 | mg |

The capsules are prepared by mixing the components and filling the mixture into hard gelatin capsules, size 1.

1-10. (canceled)

11. A method of treating a mutated-RET kinase associated disease comprising administering an effective amount of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof wherein the mutated-RET kinase associated diseases comprises thyroid cancers, pheochromocytoma, mucosal neuromas, hyperparathyroidism, parathyroid hyperplasia, Hirschsprungs disease or Cutaneous Lichen amyloidosis.

12. The method according to claim 11 wherein the thyroid cancer is selected from medullary thyroid carcinomas and papillary thyroid carcinomas.

13. The method according to claim 11 wherein the medullary thyroid carcinomas are the hereditary multiple endocrine neoplasias type 2.

14. The method according to claim 11 wherein the hereditary multiple endocrine neoplasias type 2 are MEN2A, MEN2B or FMTC.

15. The method according to claim 11, wherein the effective amount is a daily dose of 10 to 1000 mg of 4-(4-methylpiperazin-1-ylmethyl)-N-[4-methyl-3-(4-pyridin-3-yl)pyrimidin-2-ylamino)phenyl]-benzamide of formula I is administered to an adult.

16. The method according to claim 15, wherein the monomethanesulfonate salt of 4-(4-methylpiperazin-1-ylmethyl)-N-[4-methyl-3-(4-pyridin-3-yl)pyrimidin-2-ylamino)phenyl]-benzamide is administered.

17. The method according to claim 11, wherein 4-(4-methylpiperazin-1-ylmethyl)-N-[4-methyl-3-(4-pyridin-3-yl)pyrimidin-2-ylamino)phenyl]-benzamide is in mesylate salt form and in the beta crystal form thereof.
